(19) United States
(12) Patent Application Publication
(10) Pub. No.: US 2008/0102443 A1
(43) Pub. Date: May 1, 2008

(54) COLUMN CARTRIDGE, COLUMN CARTRIDGES ASSEMBLY, ANALYZER, AND ANALYZING METHOD

(75) Inventors: Masakazu FUKUDA, Kobe-shi (JP); Hiroaki TOBIMATSU, Kobe-shi (JP)

Correspondence Address: SUGHRUE MION, PLLC, 2100 PENNSYLVANIA AVENUE, N.W., SUITE 800, WASHINGTON, DC, 20037, US

(73) Assignee: SYSMEX CORPORATION, Kobe-shi (JP)

(21) Appl. No.: 11/932,312

(22) Filed: Oct. 31, 2007

(30) Foreign Application Priority Data
Nov. 1, 2006 (JP) 2006-297678

(51) Int. Cl. C12Q 1/00; B01J 19/00; G01N 30/00; C12M 1/34

(52) U.S. Cl. 435/4; 422/68.1; 422/99; 435/287.3; 73/61.55

(57) ABSTRACT

The present invention is to present a column cartridge that enables the obtainment of a plurality of analysis objects from a single sample easily. The column cartridge comprises: a first liquid sample receiving part having an opening for receiving a liquid sample; a carrier for isolating a target substance from the liquid sample; a carrier holding part for holding the carrier; and a flow path part comprising a flow path through which the liquid sample is able to pass after passing through the carrier held by the carrier holding part, and being configured to be fitted into an opening of a second liquid sample receiving part of another column cartridge.

### FIELD OF THE INVENTION

The present invention relates to a column cartridge and column cartridges assembly provided with a carrier for isolating an object substance, and an analyzer and analyzing method using the column cartridge and the column cartridges assembly.

### BACKGROUND

A column provided a carrier for isolating an object substance, and an analyzer using this column are disclosed in, for example, U.S. Pat. No. 5,918,273.

The sample column disclosed in U.S. Pat. No. 5,918,273 has a funnel for retaining a sample at the top end of the column. When a sample is put in the funnel, the top part of the funnel is sealed airtight and the sample within the funnel is pressurized. Thus, the sample is injected into the column and the analysis object (object substance) within the sample is bound to the carrier in the column.

The analyzer disclosed in U.S. Pat. No. 5,918,273 connects an inlet nozzle and an outlet nozzle to the top end of the column when analyzing the analysis object held within the column. A solvent for dissociating the analysis object from the carrier is then injected into the column through the inlet nozzle. Thereafter, the solvent which contains the analysis object is guided to a detector through the outlet nozzle, and the detection of the analysis object is performed automatically.

This analyzer provides the inlet nozzle and the outlet nozzle for each of a plurality of columns. The inlet nozzle is connected to a pump through a rotating valve, and a liquid is sent from the pump through the rotating valve and inlet nozzle to the object column by switching the flow path using the rotating valve. Furthermore, the outlet nozzle is connected to the detector through a rotating valve and the liquid is led to the detector through the rotating valve.

It is considered that when analyzing a plurality of analysis objects contained in a single sample in the analyzer which uses the column disclosed in U.S. Pat. No. 5,918,273, a single type of analysis object is bound to the carrier in the respective column by using a plurality of columns. And, it is considered that the flow path from the respective column is switched using the rotating valve to lead the analysis object held in each of the columns to the detector. Therefore, it is considered that the operation must be performed to bind the analysis objects in the sample with the carrier in the respective column by using a plurality of columns when analyzing a plurality of analysis objects contained in a single sample. Thus, a problem arises that the complexity of the operation of obtaining a plurality of analysis objects from a single sample is increased.

### SUMMARY

A first aspect of the present invention is a column cartridges assembly, comprising: a first column cartridge; and a second column cartridge, wherein the first column cartridge comprises: a first liquid sample receiving part having an opening for receiving a liquid sample; a first carrier for isolating a first target substance from the liquid sample; a first carrier holding part for holding the first carrier; and a first flow path part comprising a first flow path through which the liquid sample is able to pass after passing through the first carrier held by the first carrier holding part, and wherein the second column cartridge comprises: a second liquid sample receiving part having an opening into which the first flow path part is able to be fitted; a second carrier for isolating a second target substance from the liquid sample; a second carrier holding part for holding the second carrier; and a second flow path part comprising a second flow path through which the liquid sample is able to pass after passing through the second carrier held by the second carrier holding part.

A second aspect of the present invention is a column cartridge, comprising: a first liquid sample receiving part having an opening for receiving a liquid sample; a carrier for isolating a target substance from the liquid sample; a carrier holding part for holding the carrier; and a flow path part comprising a flow path through which the liquid sample is able to pass after passing through the carrier held by the carrier holding part, and being configured to be fitted into an opening of a second liquid sample receiving part of another column cartridge.

A third aspect of the present invention is an analyzer for analyzing a specimen using the aforementioned column cartridges assembly, comprising: a first holding part for holding the second flow path part of the column cartridges assembly; a first fluid drive part being capable of sucking a fluid containing a first liquid sample from the column cartridges assembly through the first holding part, the fluid being dispensed externally into the first liquid sample receiving part, and being capable of sending the sucked fluid to the column cartridges assembly; a second holding part for holding the first flow path part of the first column cartridge and the second flow path part of the second column cartridge separated from the column cartridges assembly which holds the first target substance on the first carrier and the second target substance on the second carrier; a dispensing part for dispensing a predetermined liquid to the first liquid sample receiving part of the first column cartridge held by the second holding part and to the second liquid sample receiving part of the second column cartridge held by the second holding part; a second fluid drive part being capable of sucking the predetermined liquid from the first and the second column cartridges through the second holding part, and being capable of sending the sucked predetermined liquid to the first and the second column cartridges; an analyzing part for analyzing a second liquid sample prepared from the predetermined liquid passing through the first carrier which holds the first target substance and a third liquid sample prepared from the predetermined liquid passing through the second carrier which holds the second target substance, and obtaining information related to the first and the second target substances; and a control part for controlling the first and the second fluid drive parts, the dispensing part and the analyzing part.

A fourth aspect of the present invention is an analyzing method for analyzing a specimen using the aforementioned column cartridges assembly, comprising: a step of dispensing a fluid containing a first liquid sample to the first liquid sample receiving part of the column cartridges assembly; a step of sucking the fluid from the column cartridges assembly, and sending the sucked fluid to the column cartridges assembly; a step of separating the first and the second column cartridges from the column cartridges assembly holding the first target substance on the first carrier and the second target substance on the second carrier; a step of dispensing a predetermined liquid to the first liquid sample receiving part of the separated first column cartridge and to the second liquid sample receiving part of the separated second column cartridge; a step of sucking the predetermined liquid from the first and the second column cartridges, and sending the sucked predetermined liquid to the first and the second column cartridges; and a step of analyzing a second liquid sample prepared from the predetermined liquid passing through the first carrier which holds the first target substance and a third liquid sample prepared from the predetermined liquid passing through the second carrier which holds the second target substance, and obtaining information related to the first and the second target substances.

### BRIEF DESCRIPTION OF THE DRAWINGS

FIG. 1 is a perspective view of the overall structure of the sample preparing device of a first embodiment of the present invention;

FIG. 2 is a perspective view showing the blank column of the first embodiment of the present invention;

FIG. 3 is a perspective view showing a multiple column of the first embodiment of the present invention;

FIG. 4 is an exploded section view of the multiple column of the first embodiment of the present invention;

FIG. 5 is a section view of a multiple column when the column is supplied to a user;

FIG. 6 is a top view showing the fixing part and the column mounting part of the sample preparing device of the first embodiment;

FIG. 7 is a front view of the fixing part and the column mounting part of the sample preparing device of the first embodiment;

FIG. 8 is a cross section view along the VIII-VIII′ line of FIG. 6;

FIG. 9 is a perspective view showing the fluid drive section of the sample preparing device of the first embodiment;

FIG. 10 is a side view (partial section view) of the fluid drive section shown in FIG. 9;

FIG. 11 is a fluid schematic diagram of the sample preparing device of the first embodiment;

FIG. 12 is a block diagram of the sample preparing device of the first embodiment;

FIG. 13 is a block diagram of the control section of the sample preparing device of the first embodiment;

FIG. 14 is a flow chart illustrating the flow of the sample preparing operation performed by the sample preparing device of the first embodiment;

FIG. 15 is a perspective view showing the overall structure of a sample preparing device of a second embodiment of the present invention;

FIG. 16 is a block diagram showing the structure of the sample preparing device of the second embodiment;

FIG. 17 is a flow chart illustrating the flow of the sample preparing operation performed by the sample preparing device of the second embodiment;

FIG. 18 is a perspective view showing the overall structure of an analyzer of a third embodiment of the present invention;

FIG. 19 is a block diagram showing the structure of the analyzer of the third embodiment; and

FIG. 20 is a flow chart illustrating the flow of the analyzing operating performed by the analyzer of the third embodiment.

### DETAILED DESCRIPTION OF THE EMBODIMENTS

The embodiments of the present invention are described hereinafter based on the drawings.

### First Embodiment

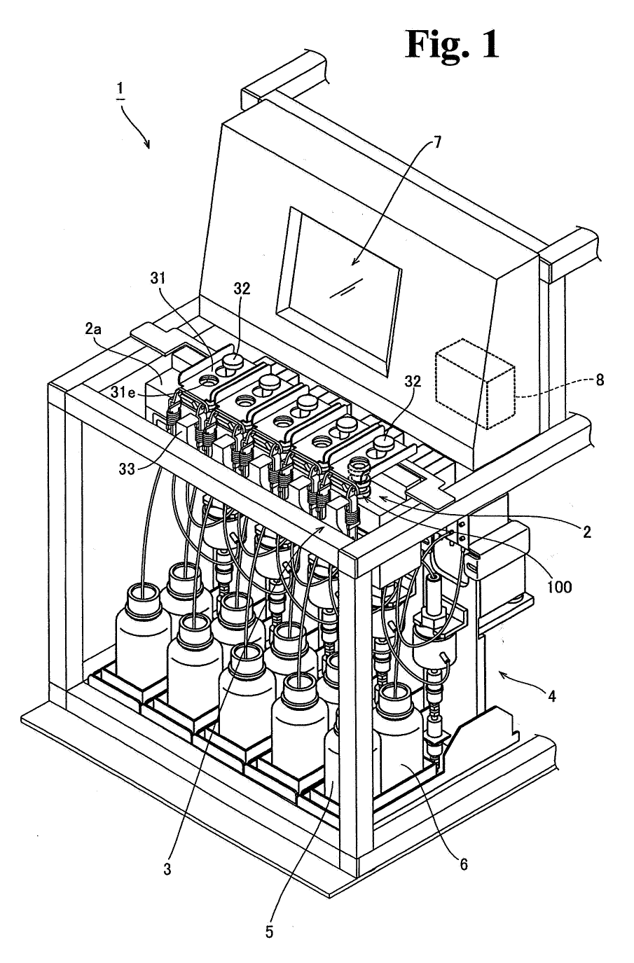

The structures of a sample preparing device 1 of a first embodiment of the present invention and a multiple column 100 used in the sample preparing device 1 are described below with reference to FIGS. 1 through 5.

The sample preparing device 1 of the first embodiment of the present invention has the function of capturing predetermined protein from an analysis object sample and performing preprocessing in which a predetermined reaction is conducted on the captured protein in an analyzer (not shown in the drawing) which measures the activity of cell cycle-related proteins used for cell cycle profiling for prognosis prediction of cancer. As shown in FIG. 1, the sample preparing device 1 performs sample preparation using a multiple column 100 in which three columns (blank column 101, first column 102, and second column 103) are connected.

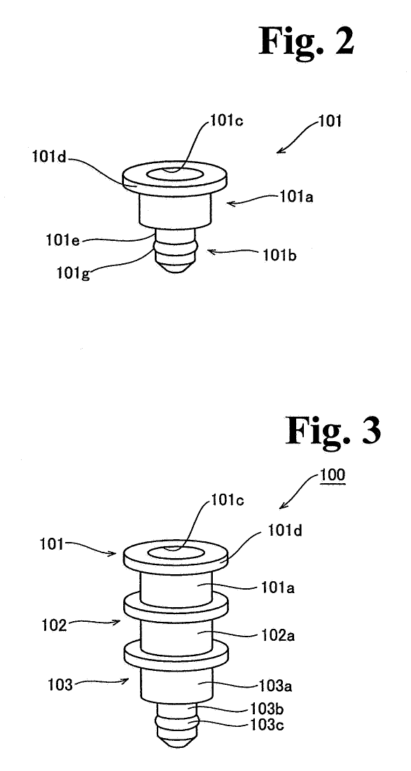

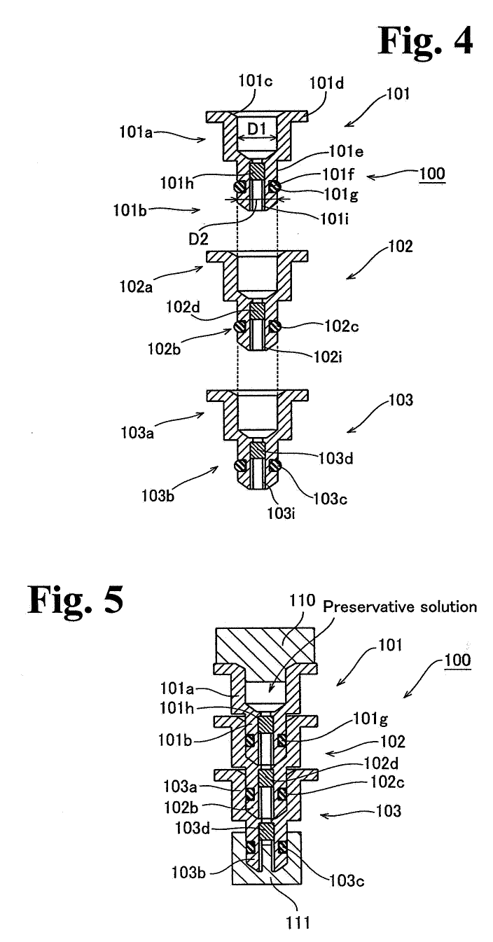

In the multiple column 100 of the first embodiment shown in FIGS. 3 and 4, the blank column 101, first column 102, and second column 103 are connected. As shown in FIG. 2, the blank column 101 includes a cylindrical receiving part 101a capable of receiving a liquid such as a sample, and a cylindrical passage part 101b disposed below the receiving part 101a and which forms a flow path through which the liquid of the receiving part 101a can flow. The receiving part 101a is configured to be capable of receiving approximately 300 μL of liquid. The receiving part 101a further includes a mouth 10c, and a flange 101d provided on the margin of the mouth 101c. The passage part 101b has a rubber O-ring 101g mounted in a groove 101f (refer to FIG. 4) provided around the circumference of the outer surface 101e. As shown in FIG. 4, the O-ring 101g protrudes slightly from the outer surface 101e of the passage part 101b. A carrier 101h, which is formed of monolithic silica (to which substance specifically bonding to the specific enzyme in the sample does not adhere), is inserted into the interior of the passage part 101b, and supported by a pipe 101i which presses against the carrier 101h. Moreover, the shape of the inner surface of the receiving part 101a and the shape of the outer surface of the passage part 101b are equivalent, that is, the inner diameter D1 of the receiving part 101a and the outer diameter D2 of the passage part 101b are configured so as to be substantially equal, or the outer diameter D2 of the passage part 101b is slightly smaller than the inner diameter D1 of the receiving part 101a so that the passage part 101b can be inserted into the interior of the receiving part 101a. The blank column 101 is used in the background measurement of a liquid such as a sample.

As shown in FIG. 4, the first column 102 is formed of the same material, shape and dimensions as the blank column 101, includes a receiving part 102a and a passage part 102b, and has an O-ring 102c mounted in the same manner as the O-ring 10g. A carrier 102d, to which substance specifically bonding to a specific enzyme (CDK1) in the sample adheres, is inserted into the interior of the passage part 102b, and supported by a pipe 102i which presses against the carrier 102d. Furthermore, the second column 103 is formed of essentially the same material, shape and dimensions as the blank column 101, includes a receiving part 103a and a passage part 103b, and has an O-ring 103c mounted in the same manner as the O-ring 101g. A carrier 103d, to which substance specifically bonding to a specific enzyme (CDK2) in the sample adheres, is inserted into the interior of the passage part 103b, and supported by a pipe 103i which presses against the carrier 103d.

The three columns of the blank column 101, first column 102, and third column 103 are connected to configure a multiple column 100 by fitting the passage part 102b of the first column 102 into the receiving part 103a of the second column 103, and fitting the passage part 101b of the blank column 101 into the receiving part 102a of the first column 102. The gap between the receiving part 103a of the second column 103 and the passage part 102b of the first column 102 is sealed by the O-ring 102c of the first column 102. Moreover, the gap between the receiving part 102a of the first column 102 and the passage part 101b of the blank column 101 is sealed by the O-ring 101g of the blank column 101.

As shown in FIG. 5, the multiple column 100 is filled with preservative solution to protect against air contact with the carriers 101h, 102d, and 103d within the multiple column 100. The multiple column 100 is supplied to the user in a sealed condition in which plug 110 is fitted into the top end of the receiving part 101a of the blank column 101 and plug 111 is fitted into the bottom end of the passage part 103b of the second column 103.

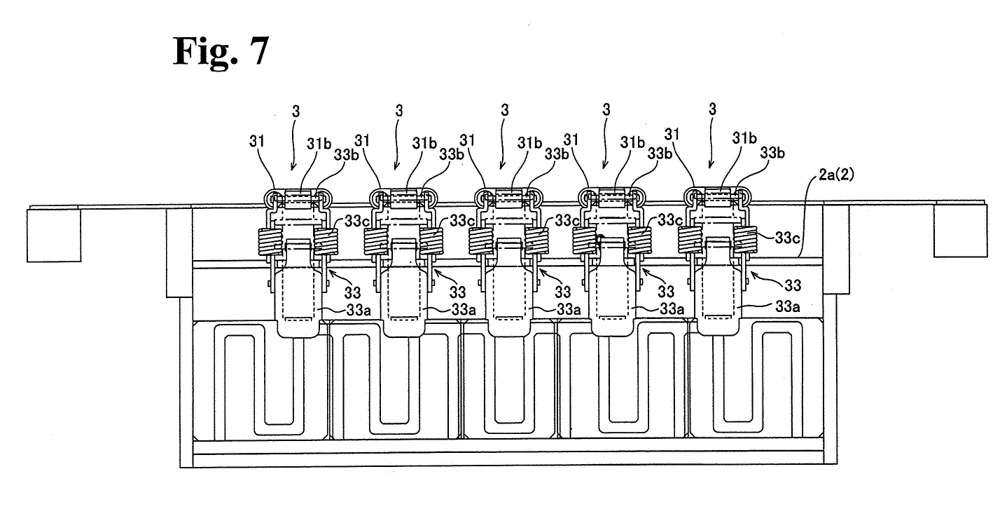

The structure of the sample preparing device 1 of the first embodiment is described below with reference to FIG. 1 and FIGS. 6 through 13.

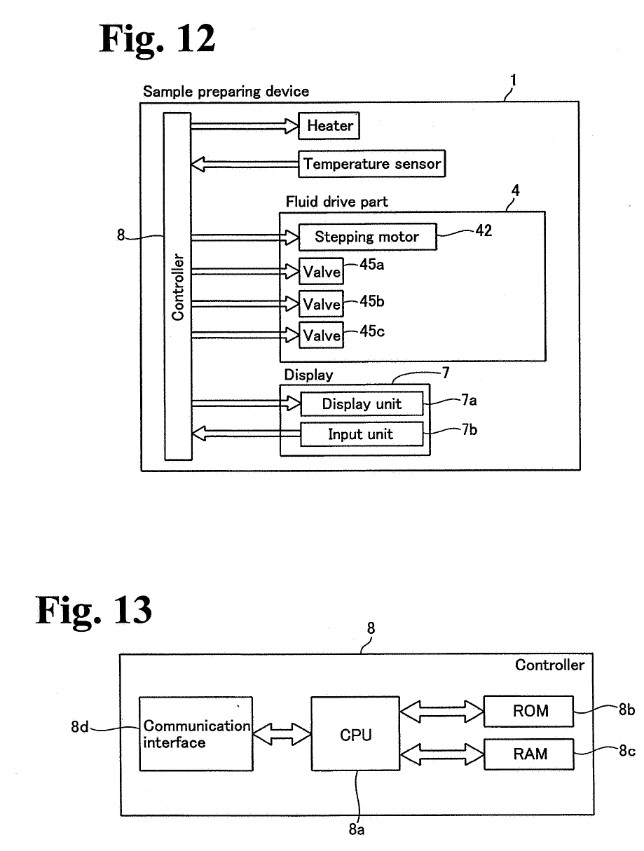

As shown in FIG. 1, the sample preparing device 1 is configured by a column mounting part 2 for installing the multiple column 100, a fixing part 3 for fixing the position of the multiple column 100 installed in the mounting part 2, a fluid drive part 4, system solution bottle 5, waste solution bottle 6, display 7, and a controller 8 for controlling the fluid drive part 4 and the display 7. A sample preparing unit is configured by the column mounting part 2, fixing part 3, fluid drive part 4, system solution bottle 5, and waste solution bottle 6. In the first embodiment, samples can be prepared using five multiple columns 100 via five sample preparation units. The column mounting part 2 is configured so as to be temperature adjustable by a heater (refer to FIG. 12) which is controlled by the controller 8 which receives signals from a temperature sensor (thermocouple; refer to FIG. 12) provided in the column mounting part 2.

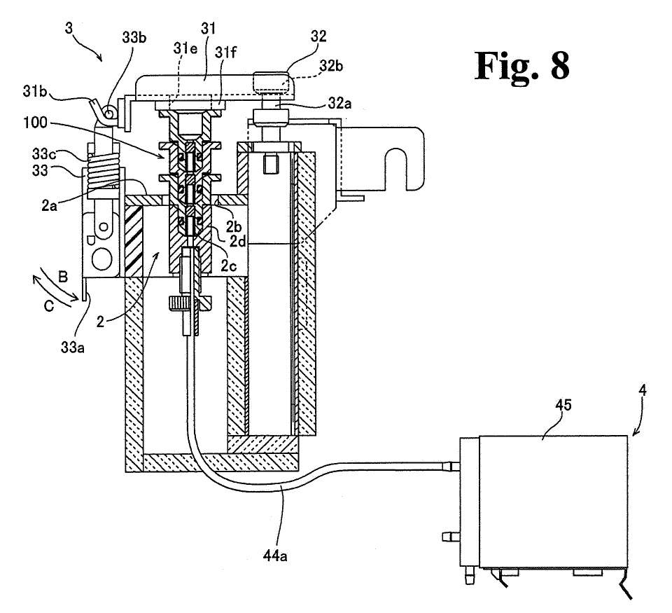

As shown in FIG. 8, the column mounting part 2 includes a column insertion hole 2b provided on the top surface of a column mounting platform 2a, and column connecting part 2d which includes a concavity 2c into which is fitted the passage part 103b of the second column 103 at the bottom end of the multiple column 100. The shape of the concavity 2c of the column connecting part 2d is equivalent to the outer surface of the passage part 103b of the second column 103, and the gap between the column connecting part 2d and the passage part 103b is sealed by the O-ring 103c when the passage part 103b has been fitted into the column connecting part 2d. Furthermore, a tube 44a of the fluid drive part 4 which is described later is connected to the bottom section of the column connecting part 2d.

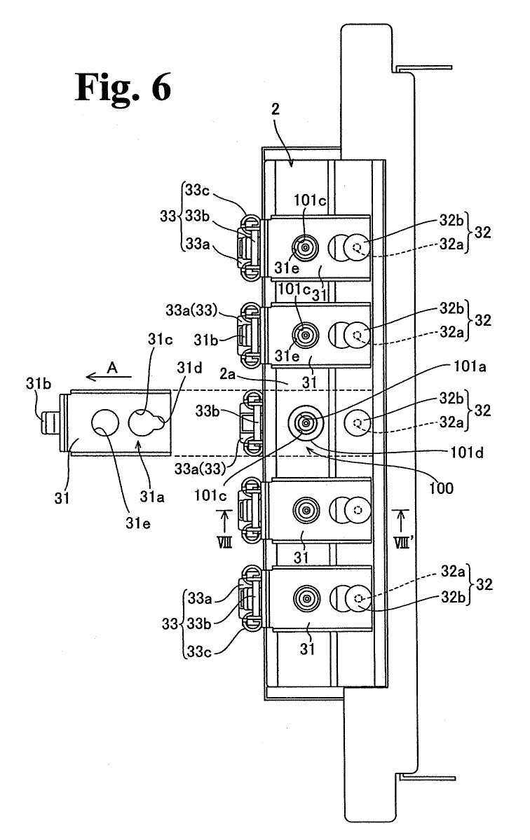

As shown in FIGS. 6 through 8, the fixing part 3 includes a pressing plate 31 which presses the top surface of the blank column 101 from above when the blank column is disposed at the top end of the multiple column, an engaging column rod 32 which engages a hole 31a (refer to FIG. 6) provided at one end of the pressing plate 31, and a chuck 33 which engages and chucks an engaging piece 31b provided at the other end of the pressing plate 31. The engaging rod 32 includes a small diameter section 32a, and a large diameter section 32b provided at the top end of the small diameter section. The pressing plate 31 is configured so that small diameter section 32a of the engaging rod 32 engages the small diameter part of the hole 31a (refer to FIG. 6) by transiting the large diameter section 32b of the engaging rod 32 from the large diameter part 31c of the hole 31a and thereafter sliding the pressing plate 31 in the arrow A direction of FIG. 6. As shown in FIG. 8, the chuck 33 includes locking part 33a which is rotatable in the arrow B direction and the arrow C direction, an engaging part 33b which engages the engaging piece 31b of the pressing plate 31, and a spring 33c for absorbing the load to the multiple column 100 applied by the pressing plate 31. The engaging part 33b is configured to move upward in conjunction with the rotation of the locking part 33a in the arrow C direction, and move downward in conjunction with the rotation of the locking part 33a in the arrow B direction. The engaging part 33b moves downward and the engaging part 31b of the pressing plate 31 moves downward in conjunction therewith when the multiple column has been fitted into the column connecting part 2d, the engaging rod 32 and engaging part 33b of the chuck 33 respectively engage the hole 31a and the engaging piece 31b of the pressing plate 31, and the locking part 33a is rotated in the arrow B direction. Thus, the multiple column is fixed in the column connecting part 2d. Furthermore, when the multiple column 100 is in this fixed condition, a hole 31e provided on the pressing plate 31 is disposed above the mouth 101c of the receiving part 101a of the multiple column 101 (blank column 101). As shown in FIG. 8, a rubber (or urethane sponge) intervenient piece 31f is mounted on the margin of the hole 31e on the bottom surface of the pressing plate 31. The multiple column 100 is fixed in place when the intervenient piece 31f presses against the flange 101d of the multiple column 100 (blank column 101).

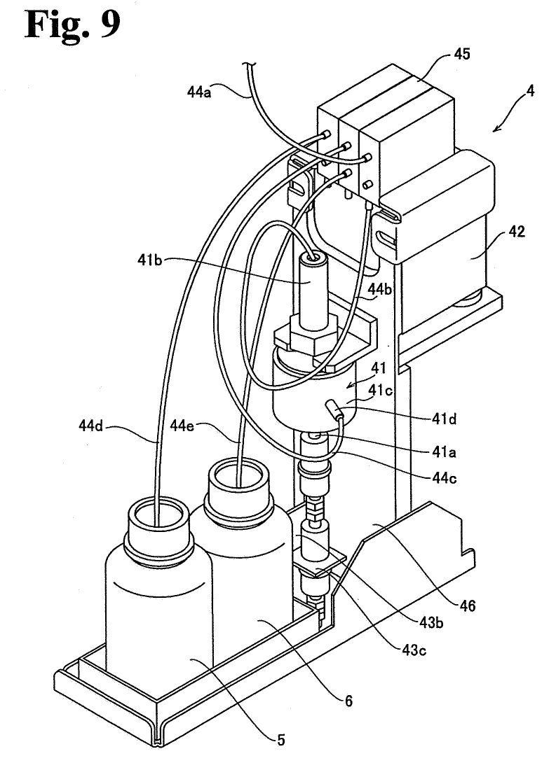

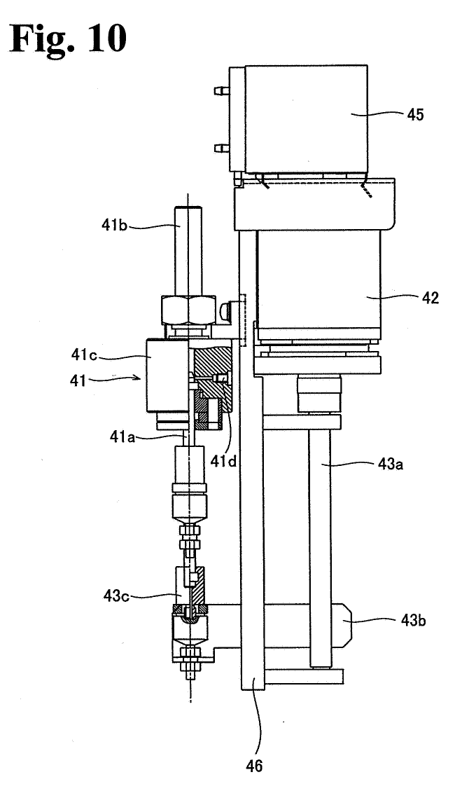

The fluid drive part 4 is provided to move the liquid such as a sample or the like which has been dispensed to the multiple column 100 so that the liquid passes through the carriers 101h, 102d, and 103d. As shown in FIGS. 9 and 10, the fluid driving part 4 includes a syringe 41 for moving liquid, a stepping motor 42 for moving the piston 41a of the syringe 41 in vertical directions, mutually fixed transfer members 43a through 43c which are provided to transfer the drive force of the stepping motor 42 to the piston 41a, a plurality of tubes 44a through 44e which form the liquid flow paths for the sample and system solution and the like (refer to FIG. 9), an electromagnetic valve 45 for switching the liquid flow path of the sample and system solution and the like, a syringe 41, and a bracket 46 for mounting the motor 42 and electromagnetic valve 45.

As shown in FIG. 10, the transfer members 43a through 43c are configured to move vertically by switching the rotation of the stepping motor 42 to drive forces in vertical directions via a female screw of the motor shaft (not shown in the drawing) and a male screw (not shown in the drawing) of the transfer member 43a into which the motor shaft is inserted. Since the piston 41a is fixedly attached to the transfer member 43c, the piston 41a is moved in vertical directions by the drive force of the stepping motor 42.

As shown in FIG. 9, the tube 44a is connected to the electromagnetic valve 45 and the column connecting part 2d (refer to FIG. 8), and the tube 44b is connected to the electromagnetic valve 45 and the tube connecting part 41b provided at the top end of the syringe 41. The tube 44c is connected to the electromagnetic valve and the tube connecting part 41d provided on the barrel 41c of the syringe 41. The tubes 44d and 44e are respectively connected to the electromagnetic valve 45 and the system solution bottle 5 and the waste solution bottle 6.

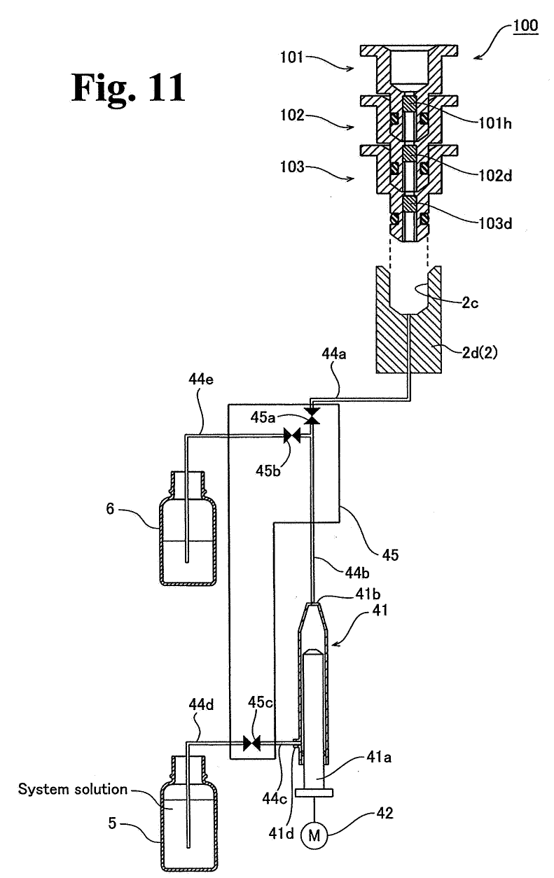

As shown in FIG. 11, the electromagnetic valve 45 has valves 45a through 45c. The valve 45a is provided to close the flow path between the column connecting part 2d and the tube connecting part 41b of the syringe 41. The valve 45b is provided to close the flow path between the waste solution bottle 6 and the tube connecting part 41b of the syringe 41. The valve 45c is provided to close the flow path between the system solution bottle 5 and the tube connecting part 41b of the syringe 41.

The display 7 is capable of displaying the content of an operation to be performed next by the user, and alerts the user to the completion of predetermined operation. As shown in FIG. 12, the display 7 is configured by a display unit 7a and an input unit 7b (touch panel), and is configured so that the controller 8 of the sample preparing device 1 can be informed of the completion of operation content when a user inputs the operation completion using the input unit 7b.

As shown in FIG. 12, the controller 8 controls the fluid drive part 4 and the display 7. The controller 8 controls the heater temperature based on signals received from a temperature sensor. The controller 8 is mainly configured by a CPU 8a, ROM 8b, RAM 8c, and communication interface 8d, as shown in FIG. 13.

The CPU 8a is capable of executing computer programs stored in the ROM 8b, and computer programs read from the RAM 8c. The ROM 8b stores computer programs which are executed by the CPU 8a, as well as data and the like used in the execution of these computer programs. The RAM 8c is used when reading the computer programs stored in the ROM 8b. The RAM 8c is also used as the work area of the CPU 8a when the computer programs are executed.

The communication interface 8d is connected to the display 7, and has the function of receiving signals indicating the completion of an operation from the display 7 when the display 7 receives the completion of a performed operation which has been input by the user. When the operation completion signals are received, the CPU 8a performs the controls for the various devices based on a computer program. The communication interface 8d also has the function of sending commands from the CPU 8a to each part in order to drive each part of the fluid drive part 4.

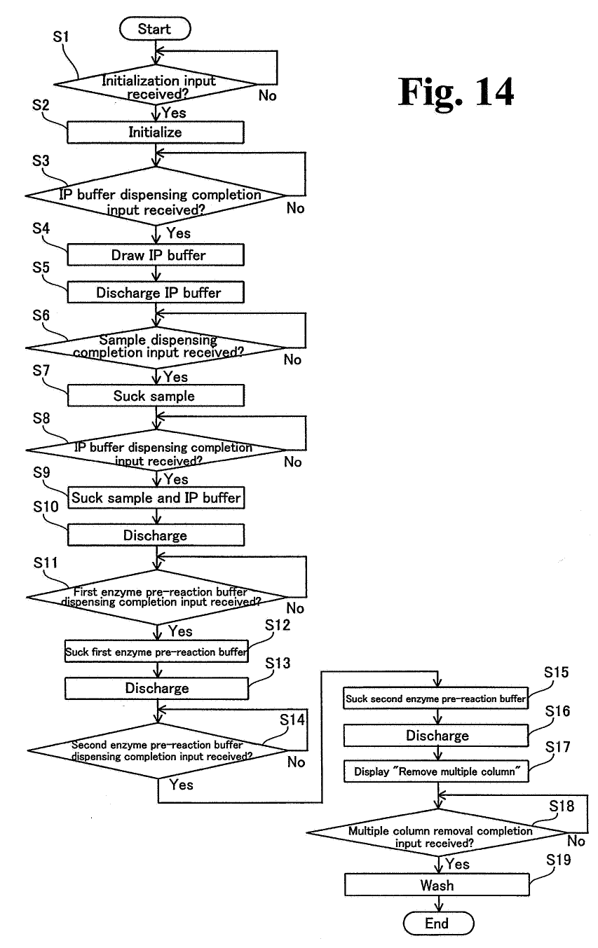

Details of the sample preparing operation using the multiple column 100 in the sample preparing device 1 of the first embodiment are described below with reference to FIGS. 11 and 14.

A user first issues an initialization command on the display 7. Then the controller 8 determines whether or not initialization input has been received in step S1 of FIG. 14. This determination is repeated when an initialization signal has not been received. The sample preparing device 1 is initialized in step S2 when an initialization signal has been received. Details of the initialization operation are described below. The valves 45a through 45c are closed when the initialization operation begins.

Liquid Inflow to the Syringe

First, the valve 45c is opened and the piston 41a down strokes as an initialization operation. Thus, system solution is inspired from the system solution bottle 5 and the system solution fills the interior of the syringe 41.

Discharge from the Syringe

Then the valve 45c is closed, the valve 45b is opened, and the piston 41a up strokes. Thus, the air and system solution within the syringe 41 are discharged to the waste solution bottle 6. The operation of the liquid inflow to the syringe and the operation of the discharge from the syringe are repeated twice thereafter.

Draining Liquid from the Column Connecting Part

Next, the valve 45a is opened and the piston 41a down strokes, and the liquid remaining within the concavity 2c of the column connecting part 2d and the liquid remaining within the tube 44a connecting the column connecting part 2d with the valve 45a is drawn into the tube 44b which connects the valve 45a with the tube connecting part 41b of the syringe 41.

Discharging Drained Liquid from the Column Connecting Part

Next, the valve 45a is closed and the valve 45b is opened, and the piston 41a up strokes. Thus, the liquid drawn into the tube 44b is discharged to the waste solution bottle 6.

Liquid Inflow to Syringe

Then, the valve 45b is closed and the valve 45c is opened, and the piston 41a down strokes. Thus, system solution is inspired from the system solution bottle 5 and the system solution fills the interior of the syringe 41.

Liquid Inflow to the Column Connecting Part (Wash)

Then the valve 45c is closed, the valve 45a is opened, and the piston 41a up strokes. Thus, system solution fills the interior of the concavity 2c of the column connecting part 2d.

Discharge from the Syringe

Then the valve 45a is closed, the valve 45b is opened, and the piston 41a up strokes again. Thus, the system solution remaining within the syringe 41 is discharged to the waste solution bottle 6.

Draining Liquid from the Column Connecting Part (Wash)

Next, the valve 45a is opened and the piston 41a down strokes, and the liquid remaining within the concavity 2c of the column connecting part 2d and the liquid remaining within the tube 44a connecting the column connecting part 2d with the valve 45a is drawn into the tube 44b which connects the valve 45a with the tube connecting part 41b of the syringe 41.

Discharging the Liquid Drained from the Column Connecting Part

Then the valve 45a is closed, the valve 45b is opened, and the piston 41a up strokes. Thus, the liquid drawn into the tube 44b is discharged to the waste solution bottle 6.

Liquid Inflow to Syringe

Then, the valve 45b is closed and the valve 45c is opened, and the piston 41a down strokes. Thus, system solution is inspired from the system solution bottle 5 and the system solution fills the interior of the syringe 41.

Liquid Inflow to the Column Connecting Part (Stand-by)

Then the valve 45c is closed, the valve 45a is opened, and the piston 41a up strokes. Thus, system solution fills the tube 44a to approximately 10 to 20 mm below the bottom of the concavity 2c of the column connecting part 2d. Filling the tube 44a with system solution to within a predetermined distance below the bottom of the concavity 2c forms an air wall (air gap) between the system solution and the in-drawn liquid when a liquid (sample or the like) is later sucked from the multiple column 100. Leakage of the sucked liquid and the system solution is thus suppressed.

Discharge from the Syringe

Then the valve 45a is closed, the valve 45b is opened, and the piston 41a up strokes again. The system solution remaining within the syringe 41 is thus discharged to the waste solution bottle 6. Thereafter, the valve 45b is closed and the valve 45a is opened. Initialization of the sample preparing device 1 is thus accomplished.

The user subsequently fits the multiple column 100 into the concavity 2c of the column connecting part 2d and fixes the column in place by the fixing part 3. The user also sucks the preservative solution retained in the receiving part 101a of the blank column 101 of the fixed multiple column 100 using a pipette or the like (refer to FIG. 5), and disposes of the preservative solution. Thereafter, 150 μL of IP buffer is dispensed into the receiving part 101a of the blank column 101 of the multiple column 100. The IP buffer is a liquid for adjusting the condition of a sample to allow the carrier to capture a target substance, that is, a predetermined enzyme (CDK1, CDK2 and the like), from the sample. The user also inputs into the sample preparing device 1 that the dispensing of the IP buffer has been completed using the display 7.

The controller 8 then determines whether or not an IP buffer dispensing completion input has been received in step S3. This determination is repeated when an input has not been received. When an input has been received, 140 μL of the IP buffer is sucked at a speed of 280 μL/min by a down stroke of the piston 41a at a predetermined speed. Thereafter, 140 μL of the sucked IP buffer is discharged at a speed of 280 μL/min by an up stroke of the piston 41a in step S5. Then a screen instructing the user to collect the IP buffer and dispense a sample is displayed on the display 7. A sample is a liquid produced by homogenizing excised cancer tissue and subjecting the homogenate to a centrifugation process.

The user then sucks and disposes of the discharged IP buffer using a pipette or the like. The user then dispenses 150 μL of the sample to the receiving part. The user also inputs into the sample preparing device 1 that the dispensing of the sample has been completed using the display 7.

In step S6 the controller 8 determines whether or not sample dispensing completion input has been received. This determination is repeated when a sample dispensing completion input has not been received. When a sample dispensing completion input has been received, the sample is sucked at a speed of 20 μL/min by a down stroke of the piston 41a at a predetermined speed in step S7. Then a screen instructing the user to collect the IP buffer and dispense a sample is displayed on the display 7.

The user then dispenses 100 μL of IP buffer to the receiving part 101a of the multiple column 100. The user also inputs into the sample preparing device 1 that the dispensing of the IP buffer has been completed using the display 7.

The controller 8 then determines whether or not an IP buffer dispensing completion input has been received in step S8. This determination is repeated when an input has not been received. When an input has been received, 100 μL of the sample and IP buffer is sucked at a speed of 20 μL/min by another down stroke of the piston 41a at a predetermined speed in step 39.

In step S10, 295 μL of the sucked sample and IP buffer is discharged at a speed of 20 μL/min by an up stroke of the piston 41a. Predetermined enzymes (CDK1 and CDK2) are respectively captured in the carrier 102d and 103d of the multiple column 100 by sucking and discharging the sample at a low speed. A screen instructing the user to collect the sample and IP buffer and dispense a first enzyme pre-reaction buffer is displayed on the display 7. This first enzyme pre-reaction buffer and a second enzyme pre-reaction buffer which is described later are washing solutions for washing the interior of the multiple column 100 to prevent a phosphorylation reaction or the like from affecting an enzyme which is subsequently captured after sample preparation has been completed.

The user then sucks and disposes of the discharged sample and IP buffer using a pipette or the like. The user next dispenses 150 μL of the first enzyme pre-reaction buffer to the receiving part 101a of the multiple column 100. The user also inputs into the sample preparing device 1 that the dispensing of the first enzyme pre-reaction buffer has been completed using the display 7.

The controller 8 then determines whether or not a first enzyme pre-reaction buffer dispensing completion input has been received in step S11. This determination is repeated when an input has not been received. When an input has been received, 140 μL of the first enzyme pre-reaction buffer is sucked at a speed of 280 μL/min by a down stroke of the piston 41a at a predetermined speed in step S12. In step S13, 140 μL of the sucked first enzyme pre-reaction buffer is discharged at a speed of 280 μL/min by an up stroke of the piston 41a. A screen instructing the user to collect the first enzyme pre-reaction buffer and dispense a second enzyme pre-reaction buffer is then displayed on the display 7.

The user sets the temperature of the column mounting part 2 at approximately 41° C. on the display 7, and after 20 minutes sucks and disposes of the discharged first enzyme pre-reaction buffer using a pipette or the like. The user next dispenses 250 μL of the second enzyme pre-reaction buffer to the receiving part 101a of the multiple column 100. The user also inputs into the sample preparing device 1 that the dispensing of the second enzyme pre-reaction buffer has been completed using the display 7.

The controller 8 then determines whether or not a second enzyme pre-reaction buffer dispensing completion input has been received in step S14. This determination is repeated when an input has not been received. When an input has been received, 240 μL of the second enzyme pre-reaction buffer is sucked at a speed of 280 μL/min by a down stroke of the piston 41a at a predetermined speed in step S15. In step S16, 240 μL of the sucked second enzyme pre-reaction buffer is discharged at a speed of 280 μL/min by an up stroke of the piston 41a. In the subsequent step S17, a screen instructing the user to collect the second enzyme pre-reaction buffer and remove the multiple column 100 is displayed on the display 7.

The user removes the multiple column 100 after sucking and disposing of the discharged second enzyme pre-reaction buffer using a pipette or the like pursuant with the instructions on the display. The user also inputs into the sample preparing device 1 that the collection of the second enzyme pre-reaction buffer and the removal of the multiple column 100 have been completed using the display 7.

The controller 8 then determines whether or not removal completion input for the multiple column 100 has been received in step S18. This determination is repeated when an input has not been received. When such input has been received, a washing operation for washing the column connecting part 2d and tubes 44a through 44e and the like are performed in step S19. The operations of the liquid inflow to the syringe and the liquid discharge from the syringe in the initialization operation of step S2 are performed as the washing operation. Thereafter, an operation including the series of draining liquid from the column connecting part, discharging liquid drained from the column connecting part, liquid inflow to the syringe, liquid inflow to the column connecting part (wash), and discharge of liquid from the syringe is performed twice. Thereafter, the operation which includes the series of draining liquid from the column connecting part (wash), discharging liquid drained from the column connecting part, liquid inflow to the syringe, liquid inflow to the column connecting part (stand-by), and discharge of liquid from the syringe is performed, whereupon the washing operation ends.

A sample is thus prepared in the sample preparing device 1 of the first embodiment.

In the first embodiment, the carrier 101h of column 101 is configured to isolate the background substances and the like of the sample, and the carriers 102d and 103d of the columns 102 and 103 are configured to isolate specific enzymes from the sample. A sample can be passed through each carrier 101h, 102d, and 103d of the three columns 101, 102, and 103 once by connecting the three columns 101, 102, and 103. Thus, the background substances and two different enzymes can easily be captured to measure the enzyme activity of a single sample.

The flange 101d is also provided on the margin of the mouth 101c of the receiving part 101a in the first embodiment as described above. Thus, when the multiple column 100 is used, the multiple column 100 can be fixed in place by the pressing plate 31 pressing against the flange 101d above the mouth 101c for receiving liquid such a sample while ensuring the flow path for injecting the sample and the like. Moreover, overflow of a sample or the like from the mouth 101c so as to spill from the multiple column 100 can be prevented by the flange 101d.

In the first embodiment described above, the gap between the passage part 101b of the blank column 101 and the receiving part 102a of the first column 102, and the gap between the passage part 102b of the first Column 102 and the receiving part 103a of the second column 103 are respectively sealed by the O-ring 102c and the O-ring 103c. When a liquid such as a sample or the like is sucked from the multiple column 100, therefore, air is prevented from entering through the gap between the passage part 101b of the blank column 101 and the receiving part 102a of the first column 102, and the gap between the passage part 102b of the first column 102 and the receiving part 103a of the second column 103. Thus, air does not come into contact with the carriers 101h, 102d, and 103d.

In the first embodiment, when supplied to a user, the passage part 103b of the second column 103 and the receiving part 101a of the blank column 101 of the multiple column 100 are sealed by plugs 110 and 111, and a preservative solution is maintained within the multiple column 100 to preserve the carriers 101h, 102d and 103d as described above. Thus, air does not come into contact with the carriers 101h, 102d, and 103d.

In the first embodiment described above, various types of liquids (sample, IP buffer, first enzyme pre-reaction buffer, and second enzyme pre-reaction buffer) are dispensed to the multiple column 100 and the piston 41a moves in vertical directions while the multiple column 100 is fitted into the column connecting part 2d. The liquid of each type can then pass through the carriers 101h, 102d, and 103d of the three columns (blank column 101, first column 102, and second column 103) of the multiple column 100. Thus, target enzymes can be captured in each of the carriers 101h, 102d, and 103d, and the multiple column 100 can be washed.

In the first embodiment described above, a system solution is moved by means of the piston 41a and switching the flow path of the system solution between the system solution bottle 5, the waste solution bottle 6, and the column connecting part 2d using the electromagnetic valve 45. The column connecting part 2d and the tubes 44a through 44e can thus be washed. After washing, the post-wash system solution can be moved to the waste solution bottle 6 for disposal by switching the flow path of the system solution to lead to the waste solution bottle 6 via the electromagnetic valve 45.

### Second Embodiment

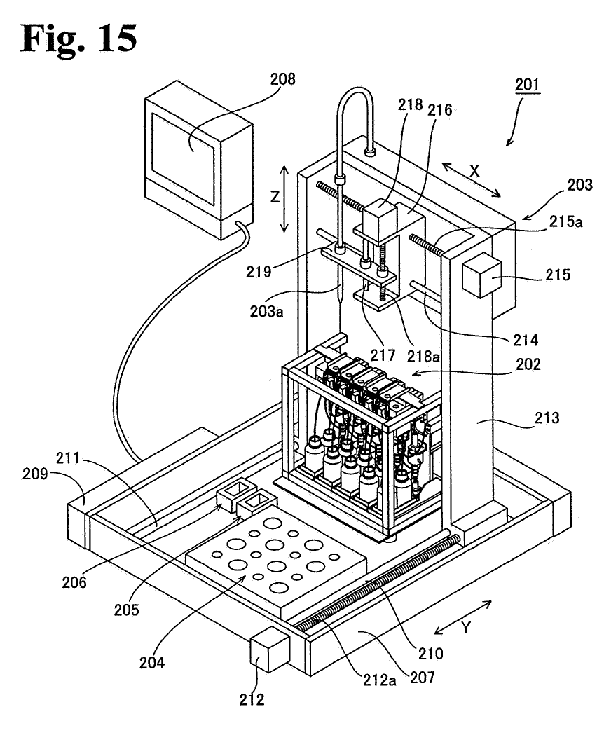

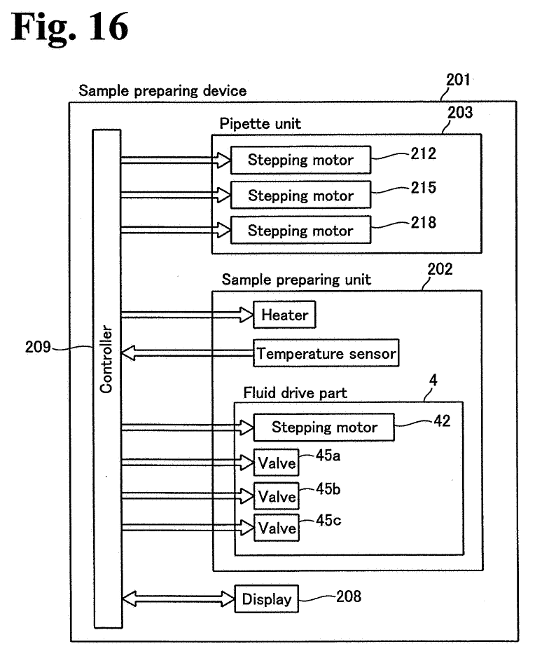

The structure of the sample preparing device 201 of a second embodiment of the present invention is described below with reference to FIGS. 15 and 16. The sample preparing device 201 of the second embodiment differs from the sample preparing device 1 of the first embodiment in that the device 201 is configured to perform the operations of dispensing, sucking, and disposing of the various liquids (sample, IP buffer and the like), which are accomplished by the user in the first embodiment, and are performed by a pipette unit 203 in the second embodiment. The multiple column 100 (blank column 101, first column 102, second column 103) of the first embodiment is used in the sample preparing device 201 of the second embodiment.

As shown in FIG. 15, the sample preparing device 201 is provided with a sample preparation unit 202, a pipette unit 203 for dispensing sucking, and disposing of the various types of liquids (sample, IP buffer, first enzyme pre-reaction buffer, second enzyme pre-reaction buffer), a liquid storing unit 204 for preserving each type of liquid, a waste unit 205 for disposing of each type of liquid, a cleaning unit 206 for cleaning the pipette 203a of the pipette unit 203, a frame 207, a display 208, and a controller 209.

The sample preparing unit 202 has a configuration which is only lacking the display 7 from the sample preparing unit 1 of the first embodiment. Detailed description is therefore omitted. The pipette unit 203 is configured so that the pipette 203a is movable in XYZ directions. Specifically, two slide shafts 210 and 211 are fixedly attached to the frame 207 so as to extend in the Y direction, and a stepping motor 212 is mounted on the frame 207 and the shaft (not shown in the drawing) of the motor 212 is connected to a ball screw 212a that extends in the in the Y direction. The slide shafts 210 and 211, and a movable block 213 into which the ball screw 212a is inserted are configured to move reciprocatingly and linearly in the Y direction via the rotational drive of the stepping motor 212. Furthermore, a slide shaft 214 is fixedly attached to the movable block 213 so as to extend in the X direction, and a stepping motor 215 is mounted on the movable block 213 and the shaft (not shown in the drawing) of the motor 215 is connected to a ball screw 215a which extends in the X direction. The slide shaft 214, and a movable block 216 into which the ball screw 215a is inserted are configured to move reciprocatingly and linearly in the X direction via the rotational drive of the stepping motor 215. Furthermore, a slide shaft 217 is fixedly attached to the movable block 216 so as to extend in the Z direction, and a stepping motor 218 is mounted on the movable block 216 and the shaft (not shown in the drawing) of the motor 218 is connected to a ball screw 218a which extends in the Z direction. The slide shaft 217, and a movable block 219 into which the ball screw 218a is inserted are configured to move reciprocatingly and linearly in the Z direction via the rotational drive of the stepping motor 218. A pipette 203a is also fixedly attached to the movable block 216. The pipette 203a is therefore configured so as to move in the XYZ directions via the rotational drives of the stepping motors 212, 215, and 218.

The waste unit 205 is provided for the disposal of liquids sucked from the multiple column 100 by the pipette 203a. The pipette 203a is configured so as to be washed in the washing unit 206 after disposing of a liquid and before sucking the next liquid. The display 8 has the same configuration as the display 7 of the first embodiment. Detailed description is therefore omitted. As shown in FIG. 16, the controller 209 controls the sample preparing unit 202 (stepping motor 42, valves 45a through 45c, and the heater), the stepping motors 212, 215, and 218 of the pipette unit 203, and the display 208. Since this configuration is identical to the controller 8 shown in the FIG. 13 of the first embodiment, further description is omitted. The controller 209 controls the heater temperature based on signals received from a temperature sensor.

The sample preparing operation performed by the sample preparing device 201 of the second embodiment is described below with reference to FIGS. 14, 15, and 17. The amount of IP buffer, sample, first enzyme pre-reaction buffer, and second enzyme pre-reaction buffer by the pipette, and the sucking and discharging speed of the piston are identical to the dispensing amounts and sucking and discharging speeds by the user in the first embodiment.

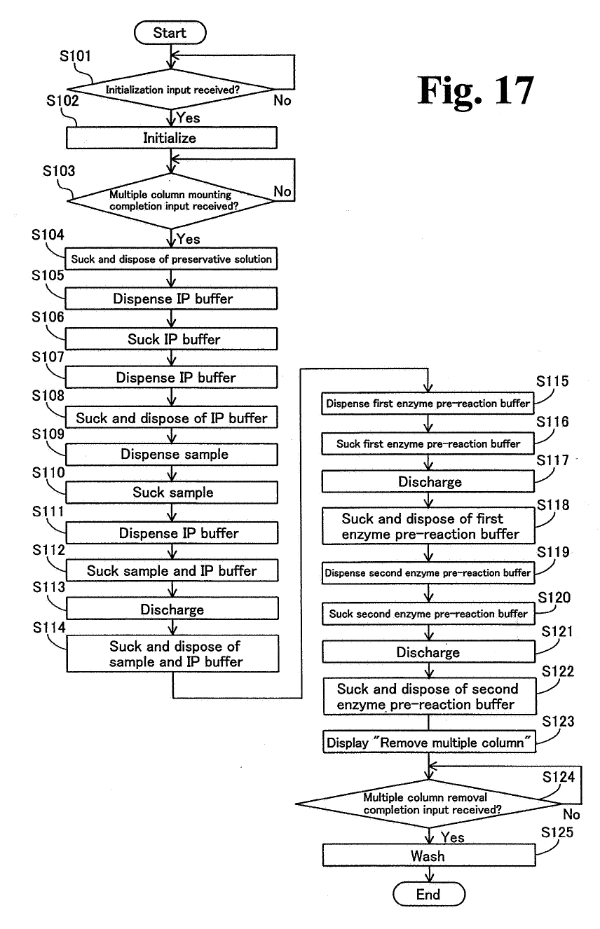

First, initialization is performed in steps S101 and S102 shown in FIG. 17 in the same manner as steps S1 and S2 (FIG. 14) of the first embodiment. Thereafter, the user installs the multiple column 100 in the sample preparing device 201, and inputs that the mounting of the multiple column 100 has been completed in the sample preparing device 201 using the display 208. The controller 209 then determines in step S103 whether or not mounting completion input for the multiple column 100 has been input. This determination is repeated when an input has not been received. When multiple column 100 mounting completion input has been received, the preservative solution stored in the multiple column 100 is sucked and disposed of by the pipette 203a of the pipette unit 203 in step S104. The pipette 203a is thereafter washed in the washing unit 206.

In step S105, IP buffer stored in a liquid storage unit 204 (refer to FIG. 15) is sucked by the pipette 203a and dispensed to the multiple column 100. In steps S106 and S107, the IP buffer is sucked and discharged by the vertical strokes of the piston 41a in the same manner as steps S4 and S5 of FIG. 14 in the first embodiment.

In step S108, the discharged IP buffer is sucked and disposed of by the pipette 203a, and the pipette 203a is washed. Then in step S109, sample stored in the liquid storing unit 204 is sucked by the pipette 203a and dispensed to the multiple column 100. The pipette 203a is thereafter washed in the washing unit 206.

In step S110, the piston 41a descends and the sample is sucked such that part of the sample remains in the same manner as step S7 of the first embodiment. In this condition, the IP buffer is dispensed from the liquid storing unit 204 to the multiple column 100 by the pipette 203a in step S111. The pipette 203a is thereafter washed in the washing unit 206.

In the subsequent steps S112 and S113, sample and IP buffer are sucked and discharged by a down stroke of the piston 41a similar to steps S9 and S10 of FIG. 14 of the first embodiment. In step S114, the discharged sample and IP buffer are sucked and disposed of by the pipette 203a, and the pipette 203a is washed in the washing unit 206.

Then in step S115, the first enzyme pre-reaction buffer is sucked from the liquid storing unit 204 and dispensed to the multiple column 100. In steps s116 and S117, the first enzyme pre-reaction buffer is sucked and discharged by the vertical strokes of the piston 41a in the same manner as steps S12 and S13 of FIG. 14 in the first embodiment. Then in step S118, the discharged first enzyme pre-reaction buffer is sucked and disposed of by the pipette 203a, and the pipette 203a is washed in the washing unit 206.

Then in step S1195, the second enzyme pre-reaction buffer is sucked from the liquid storing unit 204 and dispensed to the multiple column 100. In steps S120 and S121, the second enzyme pre-reaction buffer is sucked and discharged by the vertical strokes of the piston 41a in the same manner as steps S15 and S16 of FIG. 14 in the first embodiment. Then in step S122, the discharged second enzyme pre-reaction buffer is sucked and disposed of by the pipette 203a, and the pipette 203a is washed in the washing unit 206.

In step S123, the user is informed of the sample preparation completion and instructed to remove the multiple column 100 on the display 8. The user reads the display 8 and removes the multiple column 100. The user then inputs the completion of the removal of the multiple column 100 on the display 8.

The controller 209 then determines whether or not removal completion input for the multiple column 100 has been received in step S124. This determination is repeated when an input has not been received. When such input has been received, washing operations for washing the column connecting part 2d and tubes 44a through 44e and the like are performed in step S125 in the same manner as step S19 of FIG. 14 of the first embodiment.

Thus, sample preparation is performed in the second embodiment.

In the second embodiment described above, dispensing, sucking, and disposing of each type of liquid (sample, IP buffer, first enzyme pre-reaction buffer, second enzyme pre-reaction buffer) is performed by the pipette 203a. In addition to the effects of the first embodiment, the occurrence of human error in the timing and amount of liquid dispensed and the like is prevented compared to when the user dispenses, sucks, and disposes of each type of liquid.

### Third Embodiment

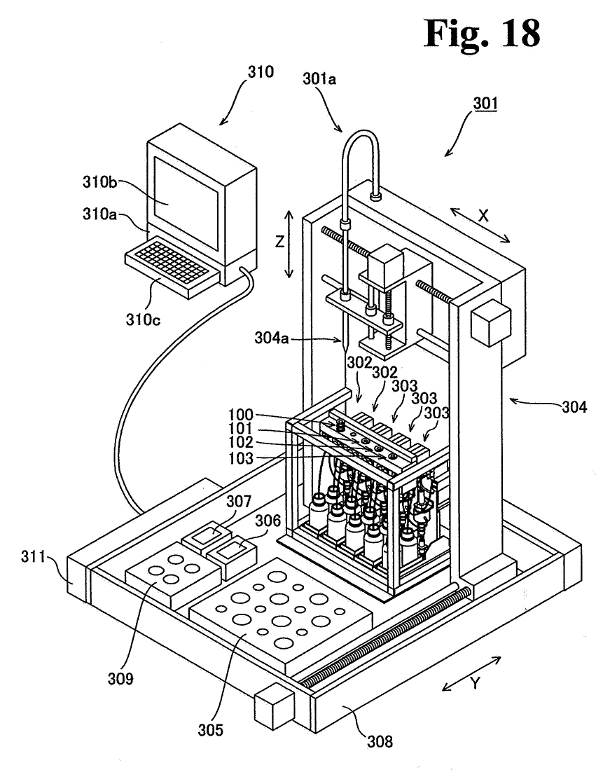

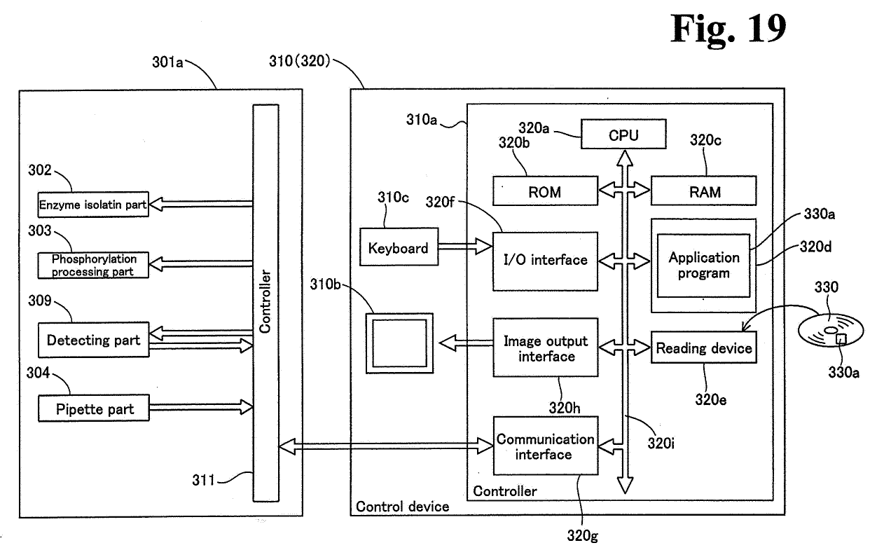

The structure of an analyzer 301 of a third embodiment is described below with reference to FIGS. 18 and 19. The analyzer 301 is configured by an analyzing unit 301a, and a control device 310 which is electrically connected to the analyzing unit 301a. The analyzer 301 of the third embodiment is provided with a detecting unit 309 and control device 310 which allows the analyzer 301 to analyze a sample after the sample has been prepared, unlike the first and second embodiments. The analyzer 301 of the third embodiment also uses the multiple column 100 (blank column 101, first column 102 and second column 103) of the first embodiment.

The analyzer 301 measures the activity of cell cycle related proteins used in cell cycle profiling for cancer prediction and prognosis. The principle of the analysis is based on the isolation of proteins (CDK1 and CDK2) related to the cell cycle present in the excised cancer tissue, inducing a phosphorylation reaction by adding protein substrate to the isolated proteins (enzymes), and binding fluorescent dye to the derived phosphate group. Then the phosphate activity of the protein (enzyme) isolated from the sample is calculated by measuring the fluorescent intensity and analyzing the measured fluorescent intensity.

As shown in FIG. 18, the analyzer 301 is provided with an enzyme isolating unit 302, a phosphorylation processing unit 303, a pipette unit 304 which includes a pipette 304a for dispensing, sucking, and disposing of each type of liquid (sample, IP buffer, first enzyme pre-reaction buffer, second enzyme pre-reaction buffer), a liquid storing unit 305 for storing each type of liquid, a waste unit 306 for disposing of each type of liquid, a washing unit 307 for washing the pipette 304a of the pipette unit 304, a frame 308, a detecting unit 309, a control device 310 configured by a personal computer, and a controller 311.

The enzyme isolating unit 302 is configured by a column mounting part 2, fluid drive part 4, system solution bottle 5, waste solution bottle 6, and an fixing part (not shown in the drawing) for attaching the multiple column 100 installed in the column mounting part 2 as shown in FIG. 1 of the first embodiment. Two enzyme isolating units 302 are provided in the third embodiment.

The phosphorylation processing unit 303 is provided to separately perform predetermined processes on each column (blank column 101, first column 102, and second column 103) of the multiple column 100 after predetermined processing in the enzyme isolating unit 302. The phosphorylation processing unit 303 is configured by a column mounting part 2, fluid drive part 4, system solution bottle 5, waste solution bottle 6, and an fixing part (not shown in the drawing) for separately attaching each column (blank column 101, first column 102, and second column 103) installed in the column mounting part 2 as shown in FIG. 1 of the first embodiment. In the third embodiment, three phosphorylation processing units 303 are provided to correspond with the three columns of the multiple column 100.

The pipette unit 304 (pipette 304a), liquid storing unit 305, waste unit 306, washing unit 307, and the frame 308 have the same structures as the pipette unit 203 (pipette 203a), liquid storing unit 204, waste unit 205, washing unit 206, and the frame 207 of the second embodiment.

The detecting unit 309 has the function of measuring the fluorescent intensity of measurement samples which have been subjected to predetermined processing in the phosphorylation processing unit 309. A container (not shown in the drawing) is provided in the detecting unit 309 to receive the measurement sample.

The control device 310 includes a controller 310a configured by a CPU, ROM, RAM and the like, as well as a display 310b, and keyboard 310c. The display 310b is provided to display analysis results and the like obtained by analyzing the digital signal data received from the detecting unit 309 through the controller 311.

The structure of the control device 310 is described below. As shown in FIG. 19, the control device 310 is a computer 320 which is mainly provided with the controller 310a, display 310b, and keyboard 310c. The controller 310a is mainly configured by a CPU 320a, ROM 320b, RAM 320c, hard disk 320d, reading device 320e, input/output interface 320f, communication interface 320g, and image output interface 320h. The CPU 320a, ROM 320b, RAM 320c, hard disk 320d, reading device 320e, input/output interface 320f, communication interface 320g, and image output interface 320h are connected by a bus 320i.

The CPU 320a is capable of executing computer programs stored in the ROM 320b, and computer programs loaded in the RAM 320c. The computer 320 functions as the control device 310 when the CPU 320a executes an application program 330a, which is described later.

The ROM 320b is configured by a mask ROM, PROM, EPROM, EEPROM or the like, and stores computer programs executed by the CPU 320a and data and the like used in conjunction therewith.

The RAM 320c is configured by SRAM, DRAM or the like. The RAM 320c is used when reading the computer program recorded in the ROM 320b and on the hard drive 320d. The RAM 320c is also used as the work area of the CPU 32a when the computer programs are executed.

The hard drive 320d contains various installed computer programs to be executed by the CPU 320a such as an operating system and application programs and the like, as well as data used in the execution of these computer programs. The application program 330a which is used in the third embodiment to measure the activity of proteins related to the cell cycle in the third is also installed on the hard disk 320d.

The reading device 320e is configured by a floppy disk drive, CD-ROM drive, DVD-ROM drive or the like, and is capable of reading the computer programs and data recorded on a portable recording medium 330. Furthermore, the portable recording medium 330 may also store the application program 330a which is used to measure the activity of proteins related to the cell cycle, such that the computer 320 is capable of reading the application program 330a from the portable recording medium 330 and installing the application program 330a on the hard disk 320d.

The application program 330a can be provided not only by the portable recording medium 330, it also may be provided from an external device that is connected to the computer over an electric communication line so as to be capable of communication by means of this electric communication line (wire line or wireless). For example, the application program 330a may be stored on the hard disk of a server computer connected to the internet, such that the computer 320 can access the server computer and download the application program 330a, and then install the application program 330a on the hard disk 320d.

Also installed on the hard disk 320d is an operating system providing a graphical user interface, such as, for example, Windows®, a product of Microsoft Corporation, U.S.A. In the following description, the application program 330a of the third embodiment operates on such an operating system.

The input/output interface 320f is configured by a serial interface such as a USB, IEEE1394, RS232C or the like, parallel interface such as SCSI, IDE, IEEE1284 or the like, analog interface such as a D/A converter, A/D converter or the like. The keyboard 310c is connected to the input/output interface 320f, so that a user can input data to the computer 320 using the keyboard 310c.

The communication interface 320g is, for example, an Ethernet® interface. The computer 320 is capable of sending and receiving data to/from the controller 311 using a predetermined communication protocol by means of this communication interface 320g.

The image output interface 320h is connected to the display 310b which is configured by configured by an LCD, CRT or the like, so that image signals corresponding to the image data received from the CPU 320a can be output to the display 310b. The display 310 displays an image (screen) in accordance with the input image signals.

The application program 330a, which is used to measure the activity of proteins related to the cell cycle and is installed on the hard disk 320d of the controller 310a, determines the activity value of proteins related to the cell cycle using the fluorescent intensity (digital signal data) pf a measurement sample received from the detecting unit 309.

The controller 311 is configured to control the enzyme isolating unit 302, phosphorylation processing unit 303, pipette unit 304, and detecting unit 309. The fluorescent intensity (digital signal data) measured in the detecting unit 309 is configured to be sent to the control device 310. A predetermined signal is sent from the controller 310a of the control device 310 to the controller 311 by an operation, such as instructing the user to start analysis, which is performed in the control device 310. This signal is received by the controller 311, which then executes the operation of each unit. The structure of the controller 311 is identical to the structure of the controller 8 shown in FIG. 13 of the first embodiment, and further description is therefore omitted.

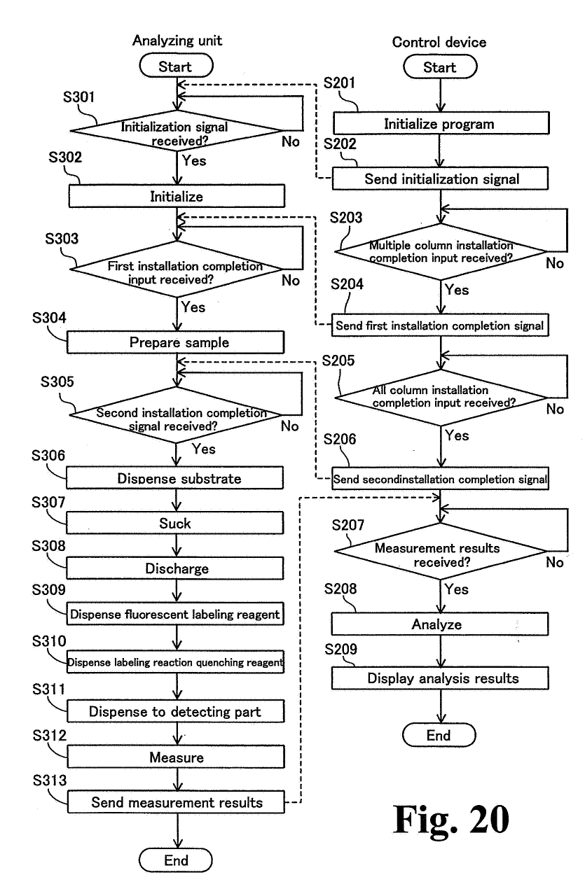

The flow of the analysis process performed by the controller 311 and the controller 310a of the analyzer 301 of the third embodiment of the present invention is described below with reference to FIGS. 17, 18, and 20.

When the user turns ON the power source (not shown in the drawings) of the control device 310, the controller 310a is initialized (program is initialized), and the power source of the analyzing unit 301a is turned ON in step S201. In step S202, the controller 310a sends the initialization signal of the analyzing unit 301a to the controller 311.

The controller 311 then determines in step S301 whether or not the initialization signal of the analysis unit 301a has been received. This determination is repeated when an initialization signal has not been received. When the initialization signal has been received, initialization is performed in step S302 which is identical to the initialization of step S102 of FIG. 17 of the second embodiment. Thereafter, the end of initialization and an instruction to install the multiple column 100 in the enzyme isolating unit 302 are displayed on the display 310b of the control device 310.

The user sees the display on the display 310b, and sets the multiple column 100 in the enzyme isolating unit 302. Then the user inputs to the control device 310 that the multiple column 100 has been installed in the enzyme isolating unit 302. The controller 310a then determines in step S203 whether or not mounting completion input for the multiple column 100 has been received. This determination is repeated when an input has not been received. When the input has been received, a first installation completion signal is sent to the controller 311 in step S204.

The controller 311 then determines in step S303 whether or not the first installation completion signal has been received. This determination is repeated when the first installation completion signal has not been received. When the first installation completion signal has been received, the enzyme isolating unit 302 captures the enzyme in step S304 in the same manner as steps S104 through S122 of the second embodiment. The user is informed of the sample preparation completion and instructed to remove the multiple column 100 via the display 310b.

After the enzyme has been isolated, the multiple column 100 is disassembled to the individual columns (blank column 101, first column 102, second column 103), and the disassembled columns are moved to the three phosphorylation processing units 303. the installation of the columns in the three phosphorylation units 302 is then input in the control device 310. In step S205, the controller 310a determines whether or not installation completion input for each column (blank column 101, first column 102, second column 103) has been received. This determination is repeated when an input has not been received. When the input has been received, a second installation completion signal is sent to the controller 311 in step S206.

The controller 311 then determines in step S305 whether or not the second installation completion signal has been received. This determination is repeated when the second installation completion signal has not been received. When the second installation completion signal has been received, a substrate solution is sucked from the liquid storing unit 305 (refer to FIG. 18) and 60 μL is dispensed to each column loaded in the phosphorylation processing unit 303 in step S306. In steps S307 and S308, the substrate solution is sucked and discharged by vertical strokes of the piston 41a. Thus, an enzyme reaction is initiated between the enzymes isolated by the carriers 101h, 102d, and 103d of each column (blank column 101, first column 102, second column 103) and the substrate in the substrate solution. The discharged solution contains a product that reflects the activity of each enzyme. The pipette 304a is thereafter washed in the washing unit 307.

In step S309, a fluorescent labeling reagent is sucked from the liquid storing unit 305 and 20 μL is dispensed to each column by the pipette 304a. Subsequently, the product which reflects enzyme activity and the fluorescent labeling reagent are reacted by standing for approximately 20 minutes. The pipette 304a is thereafter washed in the washing unit 307.

In step S310, a labeling reaction quenching reagent is sucked from the liquid storing unit 305 and 200 μL is dispensed to each column by the pipette 304a. Then the excess fluorescent labeling reagent and the labeling reaction quenching reagent are reacted by standing for approximately 3 minutes to complete the fluorescent labeling process. The pipette 304a is thereafter washed in the washing unit 307.

In step S311, the product of the fluorescent labeling process is sucked by the pipette 304a and discharged to a container (not shown in the drawings) of the detecting unit 309. In step S312, the fluorescent intensity of the product of the fluorescent labeling process held in the container is measured. In step S313, the measurement results (digital data) are sent from the controller 311 to the controller 310a, and the processing by the controller 311 ends.

Then the controller 310a determines in step S207 whether or not the measurement results have been received. This determination is repeated when the measurement results have not been received. When the measurement results have been received, the received data are analyzed by the controller 310a in step S208. Then in step S209, the analysis results are displayed on the display 310b and the processing by the controller 310a ends.

A sample is analyzed in this way in the third embodiment.

In the third embodiment, each column (blank column 101, first column 102, second column 103) of the multiple column 100 used to isolate enzymes from a sample in each carrier (101h, 102d, 103d) in the enzyme isolating unit 302 is moved to the phosphorylation processing unit 303, and phosphorylation and fluorescent labeling of the enzymes isolated in the enzyme isolating unit 302 is performed in the phosphorylation processing unit 303. Thus, the fluorescent intensity of the enzymes subjected to fluorescent labeling can be measured and the phosphorylation activity of the enzymes can be measured in the detecting unit 309.

The embodiments of the present disclosure are not to be considered limited to the examples in any aspect. The scope of the present invention is defined by the scope of the claims and not by the description of the embodiments, and may be modified insofar as such modification remains within the scope, meanings and equivalences of the claims.

For example, although the multiple column 100 is described by way of example of three connected columns of a blank column 101, first column 102, and second column 103 in the embodiments above, the present invention is not limited to this configuration inasmuch as two columns or four columns or more than four columns may also be used.

Although monolithic silica is used as a carrier installed in a column in the above embodiments, the present invention is not limited to this usage inasmuch as sepharose beads may also be used as a carrier.

What is claimed is:

1. A column cartridges assembly, comprising:
a first column cartridge; and
a second column cartridge,
wherein the first column cartridge comprises:
a first liquid sample receiving part having an opening for receiving a liquid sample;
a first carrier for isolating a first target substance from the liquid sample;
a first carrier holding part for holding the first carrier; and
a first flow path part comprising a first flow path through which the liquid sample is able to pass after passing through the first carrier held by the first carrier holding part,
and wherein the second column cartridge comprises:
a second liquid sample receiving part having an opening into which the first flow path part is able to be fitted;
a second carrier for isolating a second target substance from the liquid sample;
a second carrier holding part for holding the second carrier; and
a second flow path part comprising a second flow path through which the liquid sample is able to pass after passing through the second carrier held by the second carrier holding part.

2. The column cartridges assembly of claim 1, wherein
the first flow path part comprises a seal member on an outer circumference.

3. The column cartridges assembly of claim 1, further comprising
a third column cartridge,
wherein the third column cartridge comprises:
a third liquid sample receiving part having an opening for receiving the liquid sample;
a third carrier;
a third carrier holding part for holding the third carrier; and
a third flow path part comprising a third flow path through which the liquid sample is able to pass after passing through the third carrier held by the third carrier holding part, and being configured to be fitted into the opening of the first liquid sample receiving part.

4. The column cartridges assembly of claim 3, further comprising:
a first plug for sealing the opening of the third liquid sample receiving part; and
a second plug for sealing the second flow path.

5. The column cartridges assembly of claim 4, further comprising
a preservative solution for preserving each of the first to third carriers,
wherein the third liquid sample receiving part whose opening is sealed by the first plug, the third carrier holding part, the third flow path of the third flow path part being fitted into the opening of the first liquid sample receiving part, the first carrier holding part, the first flow path of the first flow path part being fitted into the opening of the second liquid sample receiving part, the second carrier holding part, and the second flow path sealed by the second plug, are filled with the preservative solution.

6. The column cartridges assembly of claim 1, wherein
the first carrier comprises a substance which specifically binds with the first target substance, and the second carrier comprises a substance which specifically binds with the second target substance.

7. The column cartridges assembly of claim 1, wherein
the first liquid sample receiving part comprises a flange at an upper edge.

8. A column cartridge, comprising:
a first liquid sample receiving part having an opening for receiving a liquid sample;
a carrier for isolating a target substance from the liquid sample;
a carrier holding part for holding the carrier; and
a flow path part comprising a flow path through which the liquid sample is able to pass after passing through the carrier held by the carrier holding part, and being configured to be fitted into an opening of a second liquid sample receiving part of another column cartridge.

9. The column cartridge of claim 8, wherein
the carrier comprises a substance which specifically binds with the target substance in the liquid sample.

10. The column cartridge of claim 8, wherein
the carrier is able to fix a substance which specifically binds with the target substance in the liquid sample.

11. The column cartridge of claim 8, wherein
the liquid sample receiving part comprises a flange at an upper edge.

12. An analyzer for analyzing a specimen using the column cartridges assembly of claim 1, comprising:
a first holding part for holding the second flow path part of the column cartridges assembly;
a first fluid drive part being capable of sucking a fluid containing a first liquid sample from the column cartridges assembly through the first holding part, the fluid being dispensed externally into the first liquid sample receiving part, and being capable of sending the sucked fluid to the column cartridges assembly;
a second holding part for holding the first flow path part of the first column cartridge and the second flow path part of the second column cartridge separated from the column cartridges assembly which holds the first target substance on the first carrier and the second target substance on the second carrier;
a dispensing part for dispensing a predetermined liquid to the first liquid sample receiving part of the first column cartridge held by the second holding part and to the second liquid sample receiving part of the second column cartridge held by the second holding part;
a second fluid drive part being capable of sucking the predetermined liquid from the first and the second column cartridges through the second holding part, and being capable of sending the sucked predetermined liquid to the first and the second column cartridges;
an analyzing part for analyzing a second liquid sample prepared from the predetermined liquid passing through the first carrier which holds the first target substance and a third liquid sample prepared from the predetermined liquid passing through the second carrier which holds the second target substance, and obtaining information related to the first and the second target substances; and
a control part for controlling the first and the second fluid drive parts, the dispensing part and the analyzing part.

13. The analyzer of claim 12, further comprising:
a storing part for storing a washing solution for washing a pathway of the fluid; and
a disposal part for disposing of the washing solution after it washes the pathway,
wherein the first fluid drive part comprises:
a syringe for sucking and sending the fluid, and moving the washing solution between the storing part, the disposal part and the first holding part; and
a switching part for switching a flow path of the washing solution between the storing part, the disposal part and the first holding part.

14. The analyzer of claim 12, further comprising:
a storing part for storing the fluid; and
a second dispensing part for dispensing the fluid from the storing part to the first liquid sample receiving part of the column cartridges assembly held by the first holding part.

15. The analyzer of claim 12, wherein
the control part
controls the dispensing part so as to dispense the predetermined liquid to the first and the second liquid sample receiving parts;
controls the second fluid drive part so as to suck the predetermined liquid from the first and the second column cartridges, send the sucked predetermined liquid to the first and the second column cartridges to prepare the second and the third liquid samples, send the second liquid sample to the first liquid sample receiving part and send the third liquid sample to the second liquid sample receiving part;
controls the dispensing part so as to suck the second liquid sample sent to the first liquid sample receiving part, and suck the third liquid sample sent to the second liquid sample receiving part; and
controls the analyzing part so as to analyze the second and the third liquid samples sucked by the dispensing part.

16. The analyzer of claim 12, wherein
the first liquid sample is the specimen, the first target substance is a first enzyme, the second target substance is a second enzyme, the predetermined liquid is a substrate, the second liquid sample is a product reflecting an activity of the first enzyme, the third liquid sample is a product reflecting an activity of the second enzyme, and the information related to the first and the second target substances is enzyme activity.

17. The analyzer of claim 12, wherein
the column cartridges assembly further comprises a third column cartridge,
wherein the third column cartridge comprises:
a third liquid sample receiving part having an opening for receiving the fluid;
a third carrier;
a third carrier holding part for holding the third carrier; and
a third flow path part comprising a third flow path through which the fluid is able to pass after passing through the third carrier held by the third carrier holding part, and being configured to be fitted into the opening of the first liquid sample receiving part.

18. An analyzing method for analyzing a specimen using the column cartridges assembly of claim 1, comprising:
a step of dispensing a fluid containing a first liquid sample to the first liquid sample receiving part of the column cartridges assembly;
a step of sucking the fluid from the column cartridges assembly, and sending the sucked fluid to the column cartridges assembly;
a step of separating the first and the second column cartridges from the column cartridges assembly holding the first target substance on the first carrier and the second target substance on the second carrier;
a step of dispensing a predetermined liquid to the first liquid sample receiving part of the separated first column cartridge and to the second liquid sample receiving part of the separated second column cartridge;
a step of sucking the predetermined liquid from the first and the second column cartridges, and sending the sucked predetermined liquid to the first and the second column cartridges; and
a step of analyzing a second liquid sample prepared from the predetermined liquid passing through the first carrier which holds the first target substance and a third liquid sample prepared from the predetermined liquid passing through the second carrier which holds the second target substance, and obtaining information related to the first and the second target substances.

19. The analyzing method of claim 18, wherein
the first liquid sample is the specimen, the first target substance is a first enzyme, the second target substance is a second enzyme, the predetermined liquid is a substrate, the second liquid sample is a product reflecting an activity of the first enzyme, the third liquid sample is a product reflecting an activity of the second enzyme, and the information related to the first and the second target substances is enzyme activity.

20. The analyzing method of claim 18, further comprising
a step of performing a fluorescent labeling process for the second and the third liquid samples before the step of obtaining the information related to the first and the second target substances is performed.
